(19) United States
(12) Patent Application Publication
(10) Pub. No.: US 2004/0091939 A1
(43) Pub. Date: May 13, 2004

(54) Device and method for detection of multiple analytes

(76) Inventors: To Cheung, Hong Kong (HK); Bin Li, Shanghai (CN); Yongji Peng, Shanghai (CN); Yiping Ren, Shanghai (CN); Haipeng Ge, Shanghai (CN); Fuqiao Deng, Shanghai (CN); Li Ding, Shanghai (CN); Hongmei Li, Shanghai (CN); Qifeng Cai, Shanghai (CN); Ying Chen, Shanghai (CN)

Correspondence Address: KNOBBE MARTENS OLSON & BEAR LLP, 2040 MAIN STREET, FOURTEENTH FLOOR, IRVINE, CA, 92614, US

(21) Appl. No.: 10/472,806

(22) PCT Filed: Mar. 28, 2002
(86) PCT No.: PCT/CN02/00209

(30) Foreign Application Priority Data
Mar. 28, 2001 (CN) 01105795.5
Apr. 27, 2001 (CN) 01112783.X
Jul. 11, 2001 (CN) 01113323.6
Jul. 12, 2001 (CN) 01126115.3
Aug. 14, 2001 (CN) 01126480.2
Sep. 29, 2001 (CN) 01126929.4
Sep. 29, 2001 (CN) 01126932.4
Nov. 23, 2001 (CN) 01132292.6

(51) Int. Cl. G01N 33/53; C12M 1/34

(52) U.S. Cl. 435/7.1; 435/287.2

(57) ABSTRACT

A device is provided for the detection of multiple analytes in at least one sample. The device contains a solid substrate with a test surface. On the test surface is defined at least one reaction area containing at least one array of discrete test sites. Each of these test sites have a test molecule immobilized on to it, and different test sites may have different test molecules immobilized thereon. A divider is provided for attachment onto the solid substrate. The divider contains a plurality of holes provided on an attachment surface. The attachment surface is complementary to the test surface of the solid device and is adapted for reversible attachment thereto such that when the two parts are attached, each of the holes is adjoined with a portion of the test surface to create a plurality of leak-proof chambers. The test surface within the chamber contains a plurality of test sites exposed within the chambers. Each of the chamber is preferably provided with an opening that is accessible from the exterior such that fluid introduced into the chambers may be contacted with the exposed test sites for testing. In another aspect of the present invention, a method is provided for analysing multiple analytes in the same samples.

### FIELD OF INVENTION

The present invention relates to a device and method for detecting and/or quantifying more than one analyte in a sample.

### BACKGROUND OF INVENTION

Diagnostic testing device and method for detecting various diseases and human conditions have been described in many literature and patent documents. With the advances of medical treatment and diagnostics, it has become increasingly important to monitor the state of health of a person for early diagnosis of disease conditions. The ability to test for different analytes in the same sample is a goal that many have been pursuing. Ideally, a method for analysing a single sample using a single test device should provide diagnostic information for multiple analytes in order to conserve reagents and human resources required.

U.S. Pat. No. 6,126,899 to Woudenberg et al. is directed to a method and device for simultaneously testing a sample for the presence or absence of one or more sample analytes. The device is described as having a substrate that defines a sample—distribution network having a series of chambers with each chamber having a specific reagent effective to react with an analyte. Although, multiple analytes may be analysed using the aforementioned system, the device contains numerous chambers and grooves that would require a solid substrate to be manufactured specifically for this purpose.

European Patent Application No. EP0874242 to Fitzgerald et. al. describes a solid state device for performing multi-analyte assays in which a substrate and a multiplicity of discrete reaction sites are covalently bonded with a ligand. This application discloses a device with various channels and chambers for accommodating samples to be analysed. The invention further provides an integrated analytical system for the simultaneous detection of different analytes in a multi-analyte format.

Although, the above-described systems are capable of analysing multiple analytes from a sample, there is a continual need to provide improved systems that have reduce costs for manufacturing the device and for testing the samples. There is also a need to reduce sample and reagent volume. It is therefore an object of the present invention to provide improved diagnostic systems for multiple analytes.

### SUMMARY OF INVENTION

According to one aspect of the present invention, a device is provide for the detection of multiple analytes in at least one sample. The device contains a solid substrate with a test surface. On the test surface is defined at least one reaction area containing at least one array of discrete test sites. Each of these test sites have a test molecule immobilized on to it, and different test sites may have different test molecules immobilized thereon. A divider is provided for attachment onto the solid substrate. The divider contains a plurality of holes provided on an attachment surface. The attachment surface is complementary to the test surface of the solid device and is adapted for reversible attachment thereto such that when the two parts are assembled, each of the holes is adjoined with a portion of the test surface to create a plurality of leak-proof chambers. The test surface within the chamber contains a plurality of test sites exposed within the chambers. Each of the chamber is preferably provided with an opening that is accessible from the exterior such that fluid introduced into the chambers may be contacted with the exposed test sites for testing.

In the preferred embodiment, the chamber is a well that is created with the test surface of the solid substrate acting as a base and part of the divider acting as the retaining wall such that a plurality of test sites are expose on bottom of the well within the chamber.

In another aspect of the present invention, the test molecules consist of at least one ligand and at least one antibody. The ligand is immobilized onto one test site and the antibody immobilized onto a different test site within the same chamber, such that the binding reaction of the corresponding cross-reactive analytes in the same sample is achieved simultaneously within the same reaction area.

In the preferred embodiment of the present detection device, the solid substrate is a chip with a flat top surface having a plurality of reaction areas defined thereon. Each test area contains at least one array of discrete test sites having a test molecule immobilized thereon. The divider is a sheet having holes defined by a frame with each hole corresponding to a reaction area. The frame of the sheet is adapted for reversible coupling onto the flat surface of the chip such that a leak-proof well is created therebetween. The. coupling may be done by any conventional means, including mechanical means such as, but not limited to, clips and screws, and chemical means such as glue and adhesives. Within each well, one reaction area of the top surface of the chip forms the base of the well and the corresponding parts of the frame of the sheet forms the dividing walls between the wells. The sheet is detachable such that after sample reaction, the sheet may be removed and the chip may be inserted into a standard chip reader for result reading and analysis. One advantage of this prefer embodiment is that many standard chip readers may be used to analyse test results. The removable divider sheet, when attached to the chip, creates an assembled device containing a system of wells where reactions of different samples may occur. The number of wells can be determined according to the user's requirements and the divider sheet with the required holes produced accordingly. The present invention has provided a truly versatile biochip and method for detecting multiple analytes and samples in a standard chip format.

In another aspect of the present invention, a method is provided for analysing multiple analytes in the same samples. The method comprises the steps of defining a plurality of discrete and spatially separate test site on a test surface, and immobilizing one test molecule onto each test site such that a plurality of test molecule are immobilized thereon. A fluid proof barrier around each test area is then created and a sample for analysis is added to each test area with the appropriate test reagents.

### BRIEF DESCRIPTION OF DRAWINGS

FIG. 1A is a diagram to show a top view of a solid substrate according to one embodiment of the present invention.

FIG. 1B is a diagram to show a top view of a divider sheet complementary to the substrate shown in FIG. 1A according to the same embodiment.

FIG. 1C is a diagram to illustrate the top plan view of a device according to the present invention with the top divider sheet showing FIG. 1B attached to the substrate shown in FIG. 1A.

FIG. 1D is a diagram to show a cross sectional side view across line A-A of FIG. 1C.

FIG. 1E a diagram to show a cover sheet according to the same embodiment for covering the device shown in FIG. 1C.

FIG. 2A is a diagram to illustrate the top view of a substrate according to a second embodiment of the present invention. The positions of the test sites are shown by dotted circles.

FIG. 2B is a diagram to illustrate the top view of a divider sheet complementary to the substrate shown in FIG. 2A according to the same embodiment thereof.

FIG. 2C is a diagram to illustrate the top view of the assembled device according to the second embodiment of the present invention with the divider sheet attached to the top surface of the solid substrate. The positions of the test sites are again shown by dotted circles.

FIG. 2D is a diagram to illustrate the cross sectional side view along line B-B of the device in FIG. 2C.

FIG. 3A shows a test area according to a third embodiment of the present invention.

FIG. 3B shows a test area according to the fourth embodiment of the present invention.

FIG. 4A is a diagram to illustrate the top view of a hepatitis biochip according to one preferred embodiment.

FIG. 4B illustrates a test area in a hepatitis protein chip for detecting various hepatitis antibodies of the same preferred embodiment. 60: human IgG Positive control; 62: human serum albumin (HSA) Negative control; 64: HAV Ag; 66: HCV Ag; 68: HDV Ag; 70: HBs Ag; 72: HBe Ag; 74: HBc Ag; 76: HEV Ag.

FIG. 4C illustrates a test area in hepatitis protein chip for detect various hepatitis antigens of the same preferred embodiment. 60a: HBsAg Positive control; 60b: HBeAg Positive control; 60c: AFP Positive control; 60d: HDVAg Positive control; 62: Negative control; 78: HBs Ab; 80: BBe Ab; 82: AFP Ab; 84: HDV Ab.

FIG. 4D is an example of the expected result of the diagnosis of a HAV infected patient with Anti-HAV-IgG present in the serum using the chip shown in FIG. 4A-FIG. 4C. The two test areas of the chip are shown.

FIG. 4E is an example of the expected result of the diagnosis of a HBV infected patient with serum containing HBsAg, HBeAg and HBcAb using the chip shown in FIG. 4A-FIG. 4C. The two test areas of the chip are shown.

FIG. 5A illustrates a test area of a ToRCH protein Chip according to a further preferred embodiment of the present invention. 86: Toxoplasma gondii; 88: Rubella virus; 90: CMV; 92: HSV 1; 94: HSV 2; 96: Positive control; 98: Negative control.

FIG. 5B is an example of the expected result of FIG. 5A in the diagnosis of a rubella virus infected patient using a ToRCH protein Chip. (For ease of illustration, only one test area or well is shown; test performed using a patients serum containing RV IgG)

FIG. 5C is an example of the result of FIG. 5B in the diagnosis of a CMV infected patient. (For ease of illustration, only one test area or well is shown; test performed using a patient serum containing CMV IgM)

FIG. 6A illustrates a test area of a cancer protein chip according to yet another embodiment of the present invention.

FIG. 6B is an example of the expected result of FIG. 6A in the diagnosis of a liver cancer patient using serum of the patient.

FIG. 7A illustrates a test area of autoimmune disorder protein chip.

FIG. 7B is an example of the expected result of FIG. 7A in the diagnosis of a systemic lupus erythematosus patient using the autoimme disorder protein chip. (For ease of illustration, only one test area is shown)

FIG. 7C is an example of the result of FIG. 7A in the diagnosis of a rheumatoid arthritis patient using the autoimme disorder protein chip. (For ease of illustration, only one test area is shown).

### DETAILED DESCRIPTION

As defined herein, sample includes, but is not limited to, any solution, mixture or biological fluid that contains any analytes to be tested. Sample includes positive control and negative control serum and biological fluid. A leak-proof chamber means that the chamber is capable of holding fluid in at least one direction without leakage or spillage provided that the volume of fluid is adapted to be contained within the size of the chamber. A leak-proof chamber includes enclosed structures or structures with openings. Ligand refers to any antigen or molecule that is capable of reacting with an antibody in an antibody-antigen binding reaction, a substrate in an enzyme-substrate binding reaction, or a molecule that can be recognised and bound by a receptor including but not limited to nucleic acids, chromosomal fragments, proteins, peptides, glycoprotein, lipid, polypeptides, antibiotics, steroids and other organic molecules.

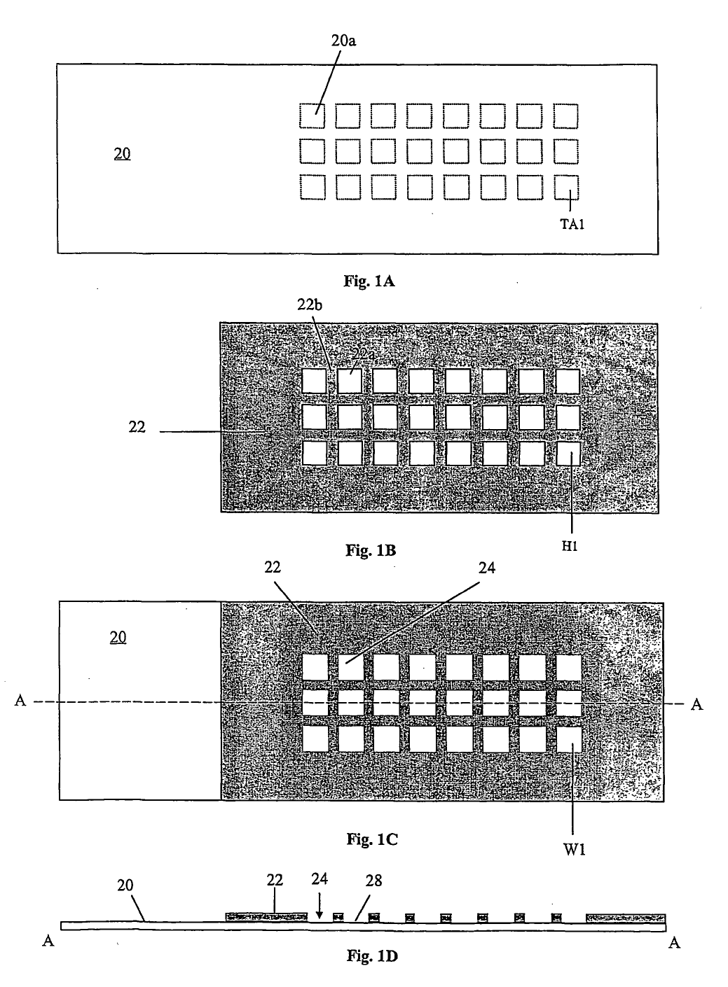

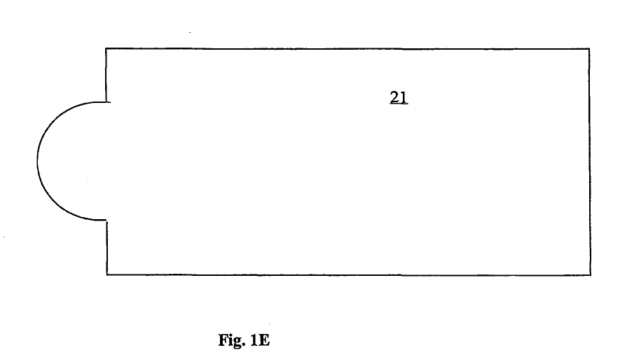

In the preferred embodiment of the present invention, and referring to FIGS. 1A to 1D, a biochip consisting of a glass slide 20 is provided with a separate top divider sheet 22. The divider sheet 22 as shown in FIG. 1B, may be made of any inert material and preferably of a flexible material such as plastic, membrane, latex, ceramic, rubber, resin, PVC or silicon. Holes 22a are punched into the divider sheet such that a frame 22b is formed around each hole. Frame 22b then acts as side walls and the top surface of the slide acts as the base to create a well or chamber 24 therebetween as shown in FIG. 1D. The size of the top divider sheet is preferably of the same dimension as or slightly shorter than the slide and contains glue on the bottom side such that it may be glued directly on the glass slide 20. In the preferred embodiment, the glue is strong enough to allow the top divider sheet to be attached to the glass slide such that wells 24 are created therebetween. The wells should be leak-proof such that when the appropriate amount of fluid is applied into each well the fluid would not flow into an adjacent well through the adjoining area. The glue should also be inert and of a material that allows the divider sheet to be detached from the slide at the convenient of the user. The number of rows or columns in each array of test sites may vary according to the user's need. The thickness of the divider sheet is dependant on the volume of reaction that is required in each well and may be determined by one skill in the art without undue experimentation. For example the sheet may be 1 to 3 mm thick. The holes and frame can be any size or shape according to the user's need. As an illustration, holes with inner dimensions from 0.3 cm×0.3 cm to 0.4 cm×0.6 cm to 1.2 cm×1.2 cm may be used. The width of the dividing wall such 22b or 34a can range from 0.15 cm to 0.6 cm. The number of holes can also be varied accordingly, such as in a 1×2, 2×3, 3×10, 3×5, 2×5, 3×8 and 4×8 array format.

In the simplest embodiment, a single test molecule may be immobilized into one test area 20a of the glass slide that correspondence to a specific well. For example, in FIG. 1B, the position of hole H1 would correspond to the position of test area TA1 as shown in FIG. 1A. One test molecule is immobilized onto test surface TA1 such that when the top divider sheet is attached and glued onto slide 20, a well W1 is created with the bottom of the well corresponding to test area TA1 as shown in FIG. 1C. In this particular example, there is only one test molecule and therefore only one test site that is found within each test area An optional cover sheet 21 as shown in FIG. 1E is also provide as a lid that can be placed onto to the outer surface of the divider to cover the solutions during incubation of the binding reaction to prevent evaporation, spillage or contamination. The cover sheet can then be removed according to the user's needs. The cover sheet may be made from any inert material such as the material used for the divider. An example is polyester film.

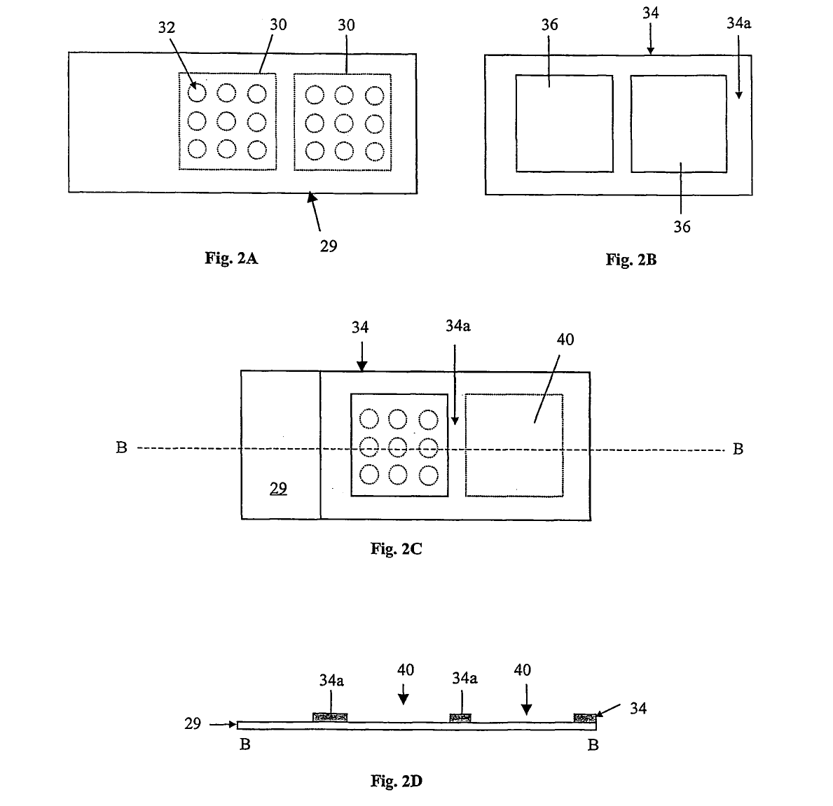

In another embodiment, a plurality of test sites are defined in each test area such that a plurality of test molecule may be found within each chamber or well. Referring first to FIG. 2A, another glass slide 29 according to the present invention is shown with two test areas 30 defined thereon. Each test area is shown as a square with dotted lines. Within each test area are test sites 32 defined in a 3×3 array and shown as circles. FIG. 2B shows a complementary divider sheet 34 with two larger holes 36 punched therein. The remainder of the sheet forms a frame 34a. FIGS. 2C & 2D show the assembled device according to this embodiment of the present invention in which two large wells 40 are formed between the divider 34 and the glass slide 29. From the illustration shown, it is clear that in this particular example, a maximum of nine different test molecules may be immobilized into the same test area and that multiple analytes may be detected using this format.

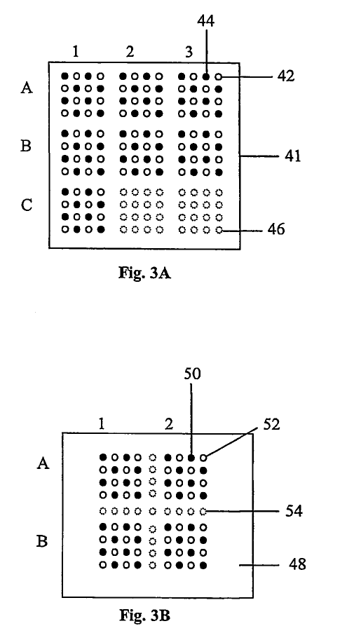

FIG. 3A shows another embodiment of the present invention in which one test area 41 (corresponding to the base of one chamber) contains nine arrays of 4×4 test sites. The other test area and the solid device are not shown for ease of illustration. It is clear that different chambers or test areas of a similar kind may be defined according to the user's requirement. Furthermore, different test areas on the same test surface may have different test site arrangements. In this example, the open circles 42 define test sites with an assay molecule immobilized thereon whilst the solid circles 44 are positive control sites with a positive control molecule immobilized thereon. The dotted circles 46 define test sites in which the negative control is immobilized thereon. The positive control molecule is expected to react with an analyte known to be present in the sample while the negative contain a test molecule that is not expected to react with any analyte in the sample. The assay molecule is one that cross reacts with an analyte (referred to as the assay analyte for ease of explanation) with unknown levels in the sample. The assay analyte is the subject of the diagnostic test. This single test area (i.e. a single well) in this example contains three rows of the 4×4 array of test sites (rows A to C) and three columns of the 4×4 arrays of test sites (columns 1 to 3). Arrays A1, A2, A3, B1, B2, B3 and C1 are identical. The positive control and assay molecule are arranged in interpolating positions. The negative control is immobilized onto test sites in Arrays C2 and C3. The entire well 41 is adapted to receive one sample such that the reaction for all the test sites can occur simultaneously and within the same reaction area. Thus in this example, three different test molecules are immobilized in the same test area for analysis. Furthermore, the multiple repetition of test sites for each test molecule allows for extremely accurate detection to occur.

Although, the examples described above show Arrays A1, A2, A3, B1, B2, B3 and C3 as having the same three assay molecules arranged in an identical format, it is clear that a different assay molecule may be immobilized onto each of these arrays in the same reaction area. For example, one assay molecule (e.g. the positive control molecule) may be immobilized into the 8 reaction sites in Array A1 while a second and different assay molecule (e.g. the negative assay molecule) is immobilized onto the 8 reaction sites in Array B3. Furthermore, it is clear that numerous different assay molecules may also be immobilized into these seven arrays depending on the number of duplicates that are required to produce the accuracy of the desired level.

FIG. 3B shows another embodiment of the present invention in which the test area 48 corresponding to one well or chamber contains a 9×9 array having a center row and a center column surrounded by four quarters. The negative control 54 is immobilized into a “cross” or cruciform configuration along the center column and center row while the positive control 50 and assay molecule 52 are arranged in interpolating positions within the four quarters. Using this format, it is clear that upon a successful experimental run in which the negative control produces no or low signal according to expectations, the four quarters would be elucidated distinctly. There is thus an advantage of this format in providing clear and unambiguous results even on simple visual inspection.

The following examples illustrate various specific embodiments of the present invention and is meant to teach one of ordinary skill in the art to make and use the present invention. These examples are meant to be illustrations only and are not intended to the limit scope of the claims as appended herewith. In the following examples, test molecules are also referred to as probes or markers. Test areas are sometimes referred to as grids and test sites as subgrids.

### EXAMPLE 1

Method of Immobilizing a Ligand onto a Test Site

Protein chip is prepared by printing and immobilized probes in a predefined grid and sub-grid pattern on microscopic solid substrate by microarrayer. The probes can be either antigens or antibodies, in the form of proteins, polypeptides or peptide, and are capable of binding to target proteins in test samples. The micro solid substrate can be derivatized glass microscopic slide, nitrocellulose membrane or silica. As much as 2390 protein spots can be printed one every 1 cm×1 cm area on glass substrate. Samples used in the reaction can be in the forms of plasma, serum, blood or other body fluid. The entire reaction procedure will be carried out in situ on the solid substrate.

Fabrication of Biochip:

1. Probes in appropriate dilution are added in individual well in microtitre plate, also known as source plate.

2. The source plate is placed in the appropriate slot in a robotic microarrayer (Prosys4510, from Cartesian Technology, CA, USA)

3. Software controls microarray pin to draw sample from the source plate.

4. Samples are printed on derivatized microscopic slide by dwelling the sample-filled pin on the surface of the slides.

5. The printed biochip is incubated at 37° C. for 1 hour to immobilize protein probes on the surface of substrate.

6. The remaining surface of substrate is blocked by Bovine Serum Albumin (BSA) and Tween 20 in TBS buffer [0.02M Tris-HCl, 0.137M NaCl, pH 7.6] for 1 h at 37° C.

7. Wash in double distilled water and dry at room temperature

8. Incubate the biochip in 0.3% sodium borohydride (NaBH₄) at 37° C. for 5 to 10 min. to reduce free aldehyde groups on glass surface.

9. Wash with double distilled water and dry at room temperature.

10. The fabricated biochip is sealed under darkness at 4° C.

Methods of Assembling the Device:

1. The outer dimension of plastic material for making multi-chamber plastic device (the divider sheet) should match that of the solid substrate.

2. Number of chambers per device can be ranging from 1×2 to 4×10.

3. The distance between the centers of each two adjoining chambers is the multiple of 4.5 mm which matches the geometry of 384 and 96 microtitre plates, pin distance of pin head of microarray and multichannel pipettmen.

4. The thickness of the plastic, determining the depth of each chamber, should be at least 1 mm and can be ranging from 1 to 3 mm.

5. One side of the multi-chamber plastic device should be sticky and can tightly adherent to the solid substrate to form leak-proof wells for the reaction. Such a device should be water-resistance, leak-proof, rigid and inert when soak in aqueous solution and used under hydraulic pressure.

6. Protein probes are microarrayed onto each chamber for biochip fabrication for multi-marker, multi-specimen usage.

7. A polyester film, which size matches the outer dimension of the multi-chamber plastic device, with single-sided adhesive is optionally used to cover on the multi-chamber biochips, forming well-isolated and enclosed chambers for the reaction.

8. The whole set up in the multi-chamber biochip can tolerate reaction temperature between −40° C. and 95° C., UV treatment and is water-resistance.

9. The bonding between the polyester film to the plastic of the multi-chamber device should be weaker than that of the device to the solid substrate. The removal of the film at each reaction step should not alter the structure of multi-chamber biochips.

10. Upon reaction completion, the plastic device can be disassembled from the solid substrates for analysis in a conventional microarray scanner (Scanarray4000, Packard Bioscience (now known as PerkinElmer Life Sciences, CT, USA).

Immobilizaiton of protein to solid substrate can also be achieved through physical interaction e.g. by charge (e.g. negative charged protein bonds with positively charge membrane or positively charged metal surface) by hydrophobic interaction (hydrophobic protein bonds to hydrophobic membrane), by detention (protein of a certain size being impeded in porous substrates such as membraneous or gel type of substrates) and through chemical interaction (e.g. bonding with epoxy slide, silanized slide, silyated slide.)

### EXAMPLE 2

Method of Detecting an Antibody in Human Serum Using a Cross-Reactive Antigen Immobilized onto a Biochip

Reaction and Detection:

1. Remove the biochip from its package.

2. Test samples (patient sample, positive and negative sera controls) are added separately to individual chambers in a biochip and allowed to react at 37° C. for 1 h.

3. Unbound reactants are washed with washing buffer with sodium salt like PBS or TBS. [PBS: 0.137M NaCl, 0.027M KCl, 0.0043M Na₂HPO₄.7H₂O, 0.0014M KH₂PO₄, pH7.3] [TBS: 0.02M Tris-Cl, 0.137M NaCl, pH7.6]

4. Remove excess buffer and allowed to dry at room temperature for 5 min.

5. Blocking solution [1% bovine serum albumin in TBS or PBS] is added to the biochip

6. Wash and dry the biochip

7. Fluorescence labeled compound (secondary antibodies or antigens) is diluted in blocking solution is added to the biochip and incubated at 37° C. for 0.5 h.

8. Wash, remove the divider sheet from the biochip and spin-dry.

9. The image is scanned by microarray scanner. Fluorescence intensity of each spot is recorded and used in data analysis and for diagnosis.

In another embodiment, steps 7 to 9 above are replaced by step 7a to 13a as described below:

7a. Biotin conjugated secondary antibody is diluted in blocking solution is added to the biochip and incubated at 37° C. for 0.5 h.

8a. Wash and dry the biochip.

9a. Streptavidin molecule in blocking solution is added and incubated at 37° C. for 0.5 h.

10a. Wash and dry.

11a. Fluorescence compounds (biotin or anti-streptavidin antibody) is diluted in blocking solution and incubated at 37° C. for 0.5 h.

12a. Wash, remove the divider sheet from the biochip and spin-dry.

13a. The image is scanned by microarray scanner. Fluorescence intensity of each spot is recorded and used in data analysis and for diagnosis.

### EXAMPLE 3

Method of Detecting an Antigen Using a Chip with a Cross-Reactive Antibody Immobilized Thereon

Reaction and Detection:

1. Remove the biochip from its package.

2. Test samples (patient sera, positive and negative sera controls) are added onto the biochip and allowed to react at 37° C. for 1 h.

3. Unbound reactants are washed with washing buffer with sodium salt like PBS, TBS. [PBS: 0.137M NaCl, 0.027M KCl, 0.0043M Na₂HPO₄.7H₂O, 0.0014M KH₂PO₄, pH7.3] [TBS: 0.02M Tris-Cl, 0.137M NaCl, pH7.6]

4. Remove excess buffer and is allowed to dry briefly at room temperature for 5 min

5. Blocking solution [1% bovine serum albumin in TBS or PBS] is added to the biochip

6. Wash and dry the biochip

7. A second primary antibody diluted in blocking solution is added to the biochip and incubated at 37° C. for 0.5 h.

8. Wash, spin-dry and block again.

9. Alkaline phosphatase (AP) or horse radish peroxidase conjugated anti-human IgG antibody (secondary antibody) diluted in blocking buffer is added and incubated at 37° C. for 0.5 h.

10. Appropriate chemiluminescent substrates (Luminol for HRP from ICN Biomedicals Inc (CA, USA) or 1,2-dioxetane-based substrate for AP from Pierce Chemical Company (IL, USA)) are added and incubated at room temparature for 1 min. (according to the manufacturer's manual)

11. The image is scanned by microarray scanner. Chemiluminescence intensity of each spot is recorded and used in data analysis and for diagnosis.

### EXAMPLE 4A

Diagnosis of Hepatitis in Human Serum

This embodiment includes the fabrication of a protein chip and its application in diagnosis for acute hepatitis, chronic hepatitis, liver cirrhosis and liver cancer that is caused by hepatitis virus infection.

Markers for hepatitis A infection diagnosis are hepatitis A antibodies (anti-HAV IgG or IgMAb); for hepatitis B are hepatitis B surface antigen (HBsAg), hepatitis B ‘e’ antigen (HBeAg), hepatitis B surface antibody (HBsAb), hepatitis B ‘e’ antibody (HBeAb) and hepatitis core antibody (HBcAb); for hepatitis C are hepatitis C antibodies (anti-HCV IgG or IgM); for hepatitis D are hepatitis D antibodies (anti-HDV IgG or IgM); and for hepatitis E are hepatitis E antibodies (anti-HEV IgG or IgM). Alpha-fetoprotein (AFP) is the marker to include in the biochip for liver cancer diagnosis. Probes or test molecules that can specifically react to these markers are printed on solid substrate during biochip fabrication.

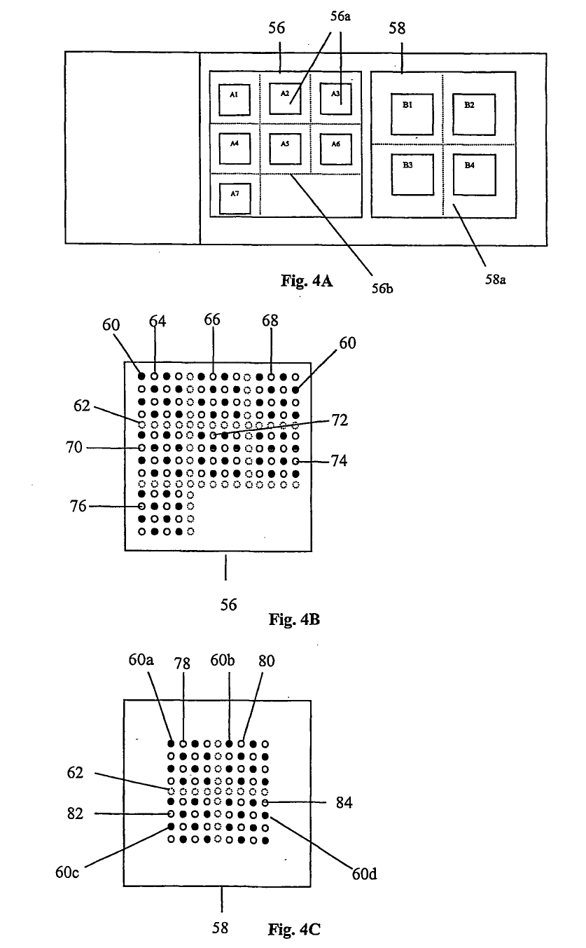

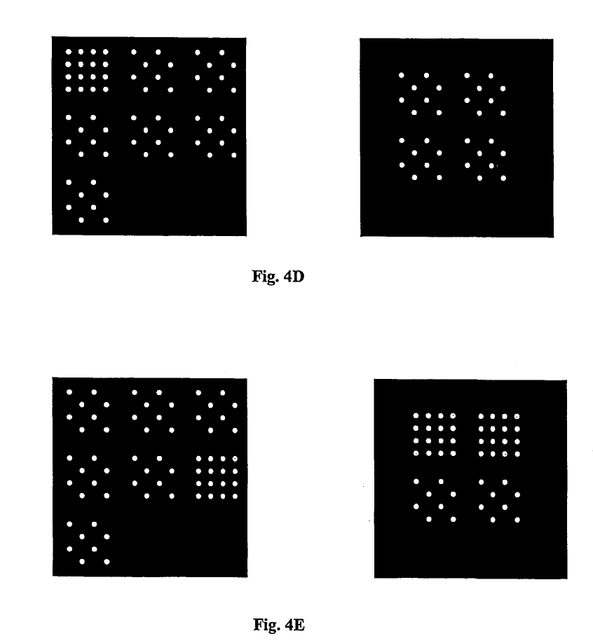

An example of a test area is shown in FIGS. 4A, 4B, 4C, 4D and 4E. This example illustrates the use of multiple repetitions provided in two test areas 56 and 58 on the same chip (FIG. 4A) for data analysis. These two test areas would serve as the bottom of two wells when an appropriate divider with two corresponding holes is placed onto this biochip for sample introduction. The advantages of this arrangement will be apparent upon explanation of the figures below. The high number of repetitions of each test molecule increases the confidence in data analysis and minimizes the geographic and machine variation during data collection, hence improving the accuracy in each biochip experiment.

Moreover, the distinct pattern as illustrated below facilitates the identification of spots in a high density array.

In this example, there are two test areas or grids 56 and 58 defined on this Biochip.

Grid or test area 56 contains seven subgrids or arrays 56a of test sites. Within each sub-grid 56a, a positive control and a assay molecule are immobilized with multiple repetitions in an array configuration. Dotted line 56b represents the positions on which multiple test sites of the negative control are defined.

Referring to FIG. 4B the test area or grid 56 is shown in greater detailed. The dotted circle 62 represent the negative controls that are found spotted in multiple repetitions across rows and columns that delineate the boundaries of each sub-grid. In each sub-grid, a positive control 60 and a test molecule (one of the hepatitis antigens) are immobilized. The test molecule immobilized into each subgrid is a different hepatitis viral antigen.

For example, in array A1, there are 16 test sites arranged in a 4×4 arrangement with the Human IgG as the positive control 60; and the test molecule hepatitis A antigen (HAV Ag) 64 immobilized in an interpolating arrangement. Six other arrays (A2-A7) are illustrated with the only difference being the test molecule immobilized thereon (HAV antigen 64, HCV antigen 66, HDV antigen 68, HBs antigen 70, HBeAg 72, HBc antigen 74 and HEV antigen 76 immobilized onto subgrids A1 to A7 respectively).

Referring now to test area 58 as shown in FIG. 4C, the test site arrangement in this area is different from that of test area 56. In test area 58, the negative control is immobilized onto multiple test sites that are arrange in a cruciform configuration across the center of the test area along line 58a of FIG. 4A. The cruciform arrangement of the negative control test sites divide the test area into four quarters. Four other arrays or subgrids (B1 to B4) are provided therearound. In array B1 as shown in FIG. 4C, the positive control hepatitis B surface antigen 60a and the test molecule anti-hepatitis B surface antigen antibody (HBs Ab) 78 are immobilized onto 16 separate and discreet test sites in an interpolating relationship. The other three quarters (B2 to B4) contain the positive control hepatitis BeAg 60b in combination with the anti-hepatitis B “e” antibody 80, positive control AFF 60c, the anti-AFP antibody 82 and the positive control hepatitis D virus antigen 60d, the anti-hepatitis D virus antibody 84 respectively.

Method for Binding Reaction:

Table 1 shows the spotting conditions for each of the test molecule on the biochip. Column 2 shows the concentration of the protein solution used for spotting while column 3 indicates the estimated size of each spot. The amount of protein that is actually immobilized onto each test site may be estimated based on the assumption that a semispherical spot is provided on each reaction site. The entire spot is allow to dry and most of the protein is considered immobilized onto the surface of the test site.

TABLE 1

| | spot | | | Time for binding |
|---|---|---|---|---|
| spots | concenstration | | spot size | reaction |
| HBsAG | 0.4 | mg/ml | 160 um | 60-90 min |
| HBsAb | 2 | mg/ml | 160 um | 60-90 min |
| HBeAg | 0.8 | mg/ml | 160 um | 60-90 min |
| HBeAb | 2 | mg/ml | 160 um | 60-90 min |
| HBcAb | 0.15 | mg/ml | 160 um | 60-90 min |
| HAV IgG | 0.8 | mg/ml | 160 um | 60-90 min |
| HCV IgG | 0.1 | mg/ml | 180 um | 60-90 min |
| HDV IgG | 0.125 | mg/ml | 160 um | 60-90 min |
| HEV IgG | 0.8 | mg/ml | 150 um | 60-90 min |

During a binding reaction patient serum is added into chambers 56 and 58 using a method similar to the one described for Example 2 and Example 3 respectively. In this example there are only two test areas shown in the biochip, one testing for hepatitis antibodies and the other for the presence of hepatitis antigen in the serum and therefore only one test serum can be tested thereon. The other test sera such as the positive and negative control sera can be applied to two separate and identical biochips in order to obtain the negative and positive results. After reaction completion, the chip is scanned and a fluorescence signal intensity from each spot is recorded.

### EXAMPLE 4B

Diagnosis of Hepatitis in Human Serum Using Multiple Wells

This example shows how the system described in Example 4A may be adapted for use in a biochip that contains multiple wells to increase the number of samples that may be tested in the same biochip. In this example, an array of three rows and eight columns such as the one shown in FIG. 1A is used for diagnosis of hepatitis in patients. For the ease of explanation, the rows of test areas are denoted as rows A, B and C from top to bottom and the columns of test areas are denoted as columns 1 to 8 respectively. In this example, the 12 test areas within columns 1 to 4 of the biochip contains test sites that are identical to the one shown in test area 56 of FIG. 4B. The 12 test areas in columns 5 to 8 each contains an array of test sites that are identical to that found in test area 58 of FIG. 4C. Using this arrangement, it can be clearly seen that for diagnosis of hepatitis antigens in the serum, there are a total of 12 test areas (i.e. wells) available. In this example, a positive control serum obtained from a patient with known hepatitis infection is added to test area A1. A negative control serum obtained from a healthy individual with no hepatitis infection is used for diagnostic reaction in well B1. The remaining 10 wells (wells A2 to A4, B2 to B4 and C1 to C4) may be used to test unknown patient sera for the presence of hepatitis antigens. In the same manner, a positive control sera is used in well A5 to provide a positive control for hepatitis antibodies while a negative control sera may be added to well B5. Their remaining wells (A6 to A8, B6 to B8, C5 to C8) may be used for testing unknown sera. The binding reaction and detection system used is the same as the one described in example 4A.

Method for Result Analysis:

R value=mean value of signal intensity of test probes falling within the 95% Gaussian distribution divided by that of positive control probe within the same subgrid.

R values from test samples in test chambers are Rs.

R value from positive control sample in positive reference chamber is Rp.

R value from negative control sample in negative reference chamber is Rn.

Rco=as ½(Rn+Rp).

There are three approaches to interpret the data

a. Method I: Individual sample with its Rs greater than 0.05 is defined as positive to the test probe; Rs between 0.02 and 0.05 is border-line and test should be repeated; and less than 0.02 is negative.

b. Method II: Individual test sample with its Rs/Rn greater than 2.1 is defined as positive, Rs/Rn between 1.5 and 2.1 is borderline; and Rs/Rn less than 1.5 is negative.

c. Method III: Individual test sample with its Rs greater than Rco is positive; and less than Rco is negative.

This invention facilitates data comparison within chamber and between chambers across the same chip. The geographic variation that may affect the signal intensity is minimized using this design in microarraying and data manipulation method.

Employing the method described above, hepatitis serum standards were obtained from the State Drug Adminstration of China (SDA) and used as test sera to check the accuracy and sensitivity of the method and device according to the present issues.

To ensure accuracy and precision of diagnostic kits in China, SDA sets the standard of sensitivity and specificity. The sensitivity test sets the weakest reactive range of diagnostic kits. The method invention according to the present passed the sensitivity test with 100% accuracy. Column 2 of Table 2 represents samples that were provided by the States Drug Administration of China as reference samples that have to be used to check the sensitivity of all diagnostic kits that are registered in China for clinical use. For HbsAg and HbsAb, a reference standard of 1 ng/ml and 10 mIU/ml are used respectively.

TABLE 2
Sensitivity test using SDA sensitivity standards

| | | Results of biochip diagnosis | | |
|---|---|---|---|---|
| | | (fluorescence intensity) | | |
| Hepatitis | SDA sensitivity | [Inventor: please verify] | | |
| Markers | reference¹ | Method I | Method II | Method III |
| HbsAg² | ≦1 ng/ml | 0.09 | 2.2 | + |
| HbsAb³ | ≦10 mIU/ml | 0.12 | 2.4 | + |
| HbeAg⁴ | 1# ≧ 1:64 | 0.10 | 2.4 | + |
| | 2# ≧ 1:128 | 0.07 | 2.2 | + |
| | 3# ≧ 1:32 | 0.10 | 2.3 | + |
| HbeAb⁵ | 55# ≧ 1:32 | 0.08 | 2.2 | + |
| | 57# ≧ 1:32 | 0.07 | 2.1 | + |
| | 61# ≧ 1:32 | 0.07 | 2.2 | + |
| HbcAb⁶ | 2# ≧ 1:32 | 0.08 | 2.2 | + |
| | 3# ≧ 1:8 | 0.11 | 2.4 | + |
| | 4# ≧ 1:16 | 0.09 | 2.2 | + |

[‘+’ is test positive,
‘−’ is test negative
¹SDA standard panel kit lot number. 9909
²HBsAg (hepatitis B surface antigen);
³HBsAb (antibody against hepatitis B surface antigen);
⁴HBeAg (hepatitis B ‘e’ antigen);
⁵HbeAb (antibody against hepatitis B ‘e’ antigen);
⁶HbcAb (antibody against hepatitis B core antigen)

TABLE 3
Specificity test using SDA standards

| | SDA specificity | | | |
|---|---|---|---|---|
| | reference (no. of | | | |
| | negative determined | | | |
| | by the current | | | |
| | method verse total | | | |
| | negative standard | Results of biochip diagnosis | | |
| Markers | analysed)¹ | Method I | Method II | Method III |
| HbsAg² | 20/20 | 0-0.018 | 0-1.38 | — |
| HbsAb³ | 20/20 | 0-0.019 | 0-1.49 | — |
| HbeAg⁴ | 15/15 | 0-0.016 | 0-1.46 | — |
| HbeAb⁵ | 15/15 | 0-0.016 | 0-1.46 | — |
| HbcAb⁶ | 15/15 | 0-0.016 | 0-1.48 | — |

Footnotes:
[‘+’ is test positive,
‘−’ is test negative
¹SDA standard panel kit lot number. 9909
²HbsAg (hepatitis B surface antigen);
³HbsAb (antibody against hepatitis B surface antigen);
⁴HBeAg (hepatitis B ‘e’ antigen);
⁵HbeAb (antibody against hepatitis B ‘e’ antigen);
⁶HbcAb (antibody against hepatitis B core antigen).

Using these three methods described above, the result are shown in the next three columns. For HbeAg, HbeAb and HbcAb, three separate unknown samples were provided by SDA and various dilutions were used to dilute the serum as indicated under column 2 (sample Nos. 1, 2 and 3 for HbeAg; Nos. 55, 57 and 61 for HbeAb; Nos. 2, 3 and 4 for HbcAb). The results of fluorescence intensity detection after the binding reaction are shown in the next corresponding column.

Table 3 shows the results of specificity test that are required by the SDA. In these test, negative sera were provided by the SDA for testing of the respective antigens or antibodies as indicated in the first column. The results are shown in the next few columns.

In column 2, the denominator of the fraction indicate the number of samples that were provided by the SDA while the numerator of the ratio represents the number of sample that were tested negative. According to SDA requirements, no false positives are tolerated. Thus, using the method according to the present invention, specificity is very high and there is little cross reactivity among patient's sera with various non-specific antigen or antibody.

FIG. 4D is an example of the expected result of a HAV infected patient. Positive signals are recorded only in test sites 64 in test area 56 and all positive controls in both test areas 56 and 58. Such a result would show that the test sera antibody is directed against HAV Ag and not other test molecules and specificity of the test is good.

FIG. 4E is an example of the expected result of a HBV infected patient. Positive signals are expected in test sites 74 in test area 56, and test sites 78 and 80 in test area 58; and also all the positive controls in test areas 56 and 58.

### EXAMPLE 5

Antenatal Testing in Pregnant Patients

This embodiment describes a biochip to be used to test the serum of pregnant women for antenatal testing purposes. Toxoplasma gondii, Rubella virus, Cytomegalovirus (CMV) and Herpes Simplex Virus I and II, collectively known as ToRCH, are routinely checked in pregnant women in which the prevalence of these diseases is high. The diseases will transmit to the fetus during pregnancy or at birth, causing severe damages to newborn infants. Because of these complications, assessment of the immune status among pregnant women and women of child-bearing age is important. Probes corresponding to antigens specific for the five ToRCH markers, together with positive and negative controls are printed onto a multi-chamber chip as described previously. The biochip allows detection of IgG and Ig simultaneously in neighbouring chambers by adding either fluorescence labeled anti-human IgG antibodies or fluorescence labeled anti-human IgM antibodies to the respective chambers. The detection of IgM in ToRCH diagnosis is to allow early diagnosis of the diseases since IgM is an antibody that is produced soon after infection, while IgG is produced after a longer lag time. However IgG levels remain high for a much longer period of time even after the infection has been cured. Hence, a high level of IgM combined with a low level of IgG in a patient's sera is indicative of an on-going acute infection.

Methods:

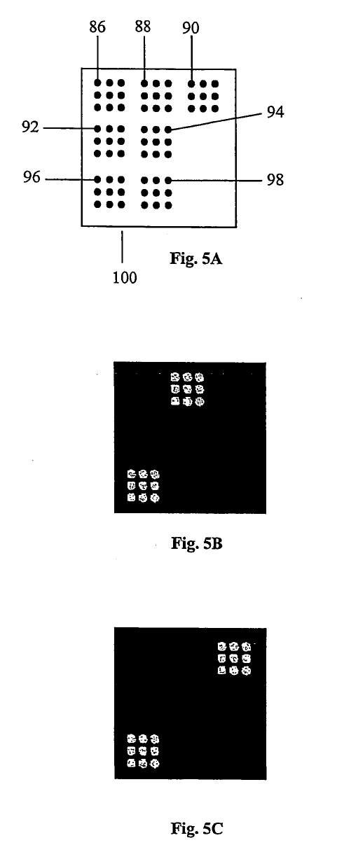

1. Probes corresponding to the antigens isolated from Toxoplasma gondii 86, Rubella virus 88, Cytomegalovirus (CMV) 90 and Herpes Simplex Virus I 92 and II 94, human IgG positive control 96 and Human Serum Albumin (IHSA) negative control 98 are printed repetitively onto test sites that are arranged in a 3×3 array or as shown in FIG. 5A. These different subgrids are defined within the same test area 100 that will be found in a single well when an appropriate divider is attached thereon. In the preferred embodiment, the biochip in this example contains eight test areas that are identical to the one shown in FIG. 5A. Two tested areas are used for incubation with a positive serum while two others are used for the negative control serum. Two additional patient's sera can then be tested in this biochip in duplicates.

2. Immobilization, drying, blocking, reaction, washing are carried out as described in Example 2.

3. To detect ToRCH IgG in serum, fluorescence labeled anti-human IgG antibodies are used in one of the wells that has been incubated with the serum as indicated above.

4. To detect ToRCH IgM, fluorescence labeled anti-human IgM antibodies are added to the duplicate well that has been incubated previously with the respectively serum.

5. FIG. 5B shows the experimental results of an assay using serum from a rubella virus infected patient. Positive signal was detectable in test molecule 88 and positive control 96.

6. FIG. 5C shows the experimental results of an assay using serum of a CMV infected patient. Positive signal was detectable in test molecule 90 and positive control 96.

### EXAMPLE 6

Use of the Streptavidin/Biotin System as a Universal Amplification System

This embodiment uses the fluorescence/enzyme labeled streptavidin and biotin method (FLSAB/ELSAB) as an enhanced reporting system in protein chip analysis. In the current protein chip technology, fluorescence or enzyme conjugated antibodies are commonly used in the reporting system. This embodiment introduces a signal amplification step to the present reporting system. Sensitivity of protein chip increases by ten folds when using the amplification system. Therefore, less amount of probes may be used in printing. In return, background is reduced, signal to noise ratio increases and the chance of false positive results is minimized. It also facilitates the omission of a difficult step on labeling of fluorescence dye to acid protein.

Fabrication of Protein Chip:

1. Arraying of protein probes on solid substrates such as aldehyde slides, nitrocellulose or nylon membrane.

2. Protein probes on slides are dried to fix for overnight at room temperature or 1 hour at 37° C.

3. Substrate surface is blocked by blocking buffer containing bovine serum albumin (BSA) and tween 20 for 1 hour at 37° C.

4. Rinse with double distilled water and store dried at room temperature.

Methods:

1. Specimen sample is added into each chamber of protein chip and incubated at 37° C., 1 hour.

2. Excessive specimen is washed with washing buffer and spun dry.

3. The chip is blocked with blocking buffer and washed with washing buffer as described before.

4. Wash and spin dry.

5. Biotin conjugated biomolecules which bind specifically to the target markers are diluted in blocking buffer, added into each chamber and incubated for 30 min at 37° C.

6. Wash and spin dry.

7. Streptavidin is diluted in blocking buffer, added to the chip and incubated for 30 min at 37° C.

8. Wash and spin dry

9. Enzyme alkaline peroxidase (AP) or horse raddish peroxidase (HRP), or fluorescence conjugated biotin diluted in blocking buffer is added to the chip and incubated for 30 min at 37° C. in darkness.

10. If enzyme conjugated biotin is used in (9), appropriate chemiluminescent substrates, Luminol, Pierce Chemical Company (EL, USA) for HRP or 1,2-dioxetane-based substrate for ICN Biomedicals Inc. (CA, USA), are added and incubated at room temperature for 1 min. (according to the manufacturer's manual) After completion, the chip is washed, divider sheet is removed, and chip was spun dry and scanned with micoarray scanner.

### EXAMPLE 7

Method and Device for Cancer Diagnosis

This embodiment describes a high throughput and sensitive diagnostic method and biochip for cancers. This chip allows the detection of cancer at early stage and at the same time, determines the type and subtype of caner. Probes are antibodies corresponding to tissue specific tumor marker; positive controls are the corresponding tumor markers. Human serum albumin is the negative control.

Cancer chips are categorized according to the organ/tissue type. Each type of cancer chip consists of a panel of antibodies that react to tissue specific tumor markers. The expression profile in each antibody panel allows further subtyping of the cancer. For example, γ-GT I′, II II′ are found only in liver caner; Concanavalin A reactive alpha-fetoprotein (R Con A AFP) are predominantly found in serum samples of primary liver cancer while in secondary liver cancer, gonadal and extra gonadal germ cell tumors had a significant reduction of R ConA AFP with elevation of nonreactive form. Therefore, the use of panels of tumor markers in cancer biochips will facilitate cancer diagnosis, typing and subtyping.

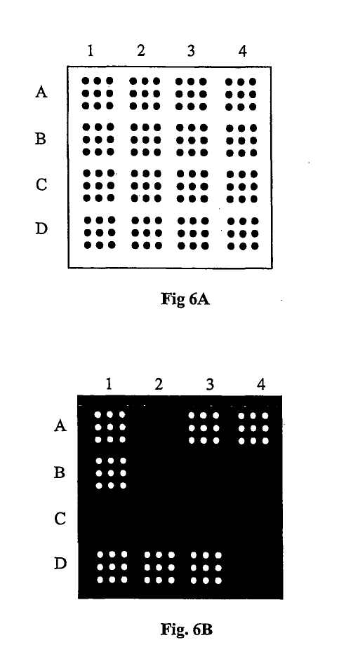

FIG. 6A shows one test area in a cancer chip with 16 arrays (A1 to D4). Each array contains 9 discrete test sites with the same test molecule immobilized thereon. Array A1 has test molecule anti alpha-fetoprotein (AFP) antibody printed thereon; A2 is printed the anti gamma-glutamyl transferase (γ-GT) isozyme I antibody, A3 is printed the anti gamma-glutamyl transferase (γ-GT) isozyme I′ antibody; A4 is printed the anti gamma-glutamyl transferase (γ-GT) isozyme II antibody; B1 is printed the anti gamma-glutamyl transferase (γ-GT) isozyme II′ antibody; B2 is printed the anti des-gamma carboxy prothrombin (DPC) antibody; B3 is printed the anti alpha-L-fucosidase antibody; B4 prints the anti 5′ nucleotide phosphodiesterases isozyme V antibody; C1 prints the anti glutathione S-transferase-placental form antibody; C2 prints the anti apha-1-antitrypsin antibody; C3 prints the anti ferritin antibody; C4 prints the anti acidic isoferritin (AIF) antibody; D1 prints the anti hepatitis B surface antigen antibody; D2 and D3 print the positive controls corresponding to the 13 tumour markers and also human IgG; and D4 prints the negative control.

FIG. 6B shows an example of the kinds of the diagnostic result that can be obtained from a liver-cancer patient, for example with positive signals detected in array A1, A3, A4, A5 and D1, and positive controls D2 and D3.

### EXAMPLE 9

Diagnosis of Autoimmune Disorder

Autoimmune diseases are characterized by the presence of high levels of circulating IgM and IgG autoantibodies in patient serum. There are two categories of autoimmune diseases: single organ or cell type, and systemic type. Example of the former type are autoimmune hemolytic anemia (AIHA) in which the body attacks its own red blood cells (RBC) with elevated level of IgG autoantibody and IgM antibody; Myasthenia gravis in which patient suffers from severe muscular weakness with elevated in autoantiodies to acetylcholine receptors; and Hashimoto thyroiditis in which anti-thyroglobin antibody and antimicrosomal antibody are elevated. Examples of the later are systemic lupus erythematosus (SLE) in which antinuclear ribonucleoprotein antibody (anti-dsDNA and anti-SM) are detected; and rheumatoid arthritis in which rheumatoid factor is detected.

Methods:

1. Autoimmune protein chip is to detect autoantibodies in sera of patients. Probes corresponding to antigens of autoimmune disease markers are printed on the multi-chamber chip for fabrication. The markers are antinuclear ribonucleoprotein antibody, rheumatoid factor, anti acetylcholine receptor antibody, anti red blood cell antibody, anti thyroglobulin antibody and antimicrosomal antibody.

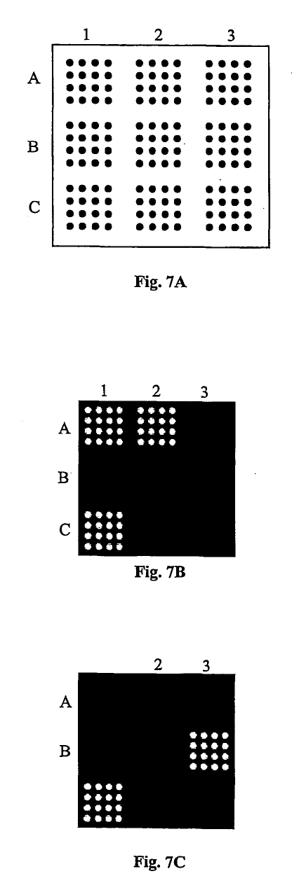

Example of the design of autoimmune diagnosis protein chip is illustrated in FIG. 7A. Within the same test area (representing one well or chamber in the assembled device), 9 discrete arrays (A1 to C3) are defined. Each array is immobilized one type of molecule which is different from the other arrays. Within the same array, the 16 test sites (4×4) contain the identical test molecule. A1 prints the double stranded DNA molecule; A2 prints the SMITH antigen; A3 prints the acetylcholine receptor; B1 prints thyroglobulin; B2 prints microsomal antigen; B3 prints abnormal IgG; C1 prints the human IgG positive control; C2 prints the negative control; and C3 prints red blood cell. SMITH is a complex of RNA and non-histone nuclear protein that helps DNA stay in its correct shape; it is released into the blood stream together with DNA upon cell lysis and is a marker for SLE.

2. Autoimmune protein chip is an indirect immuno-based assay. Patient sera are added into individual chamber. As in the previous example 8, each serum may be added to duplicate chambers for the detection of IgG as compared to the detection of IgM. Autoantibodies in the serum react with the stationary phase probes. After washing, a fluorescence labeled anti-human IgG antibody is added to the chamber for detection of cross-reaction IgG.

3. For the detection of cross-reaction bivalent or multivalent IgM, a double-antigen-sandwich method can also be applied in the autoimmune protein chip. After the reaction with serum, fluorescence label antigens are added to the reaction chamber for detection.

4. After reaction completion, biochips are scanned by a microarray scanner.

FIG. 7B shows an example of the diagnostic result of a systemic lupus erythematosus patient. Positive signals will appear in test sites A1 and A2, and positive control site, C1.

FIG. 7C shows an example of the diagnostic result of rheumatoid arthritis patient. Positive signals will appear in test site B3 and positive control C1.

The detailed description for the specific embodiment above have been presented for the purposes of illustration and explanation. It is not intended to be exhaustive or to limit the invention to the precise forms disclosed, as many modifications and variations are possible in the light of the teaching provided. For example, it is clear that the arrangement shown in, FIGS. 4A to 4C is for illustration only and that smaller test areas may be defined onto the same biochip. As an variation, three duplicates of test areas 56 and 58 may be provided on the same chip such that the test serum and the positive and negative sera may also be tested on same biochip. Furthermore, additional test area may also be defined on the biochip in combination with a suitable divider with the corresponding number of holes such that multiple patients' samples may be tested on the same biochps.

As a further example, the use of anti-human IgM and anti-human IgG antibodies for the diagnosis of acute infections by analysing the antibody profile of the patient may be applied to many diseases in addition to the one disclosed in example 5.

1. A device for detecting multiple analytes comprising a solid substrate and a divider,
said substrate having a test surface with a plurality of discrete test sites defined thereon, each said test site having a test molecule immobilized thereon;
said divider having a plurality of holes at predetermined positions provided on an attachment surface, said attachment surface complementary to said test surface and adapted for attachment thereto, said divider reversibly attachable onto said solid substrate such that each said hole is adjoined with a portion of said test surface to create a plurality of leak-proof chambers, said test surface within said chamber having a plurality of test sites exposed therein, said chamber further having an opening that is accessible from the exterior such that fluid introduced into said chamber may be contacted with said exposed test sites for testing.

2. A device according to claim 1 where in said test molecule is an antigen or an antibody.

3. A device according to claim 1 where in a cover is provided, said cover having a covering surface adapted for closing said opening of said chamber to prevent spillage or contamination between solutions in different chambers.

4. A device according to claim 3 herein said cover is a polyester sheet.

5. A device for detection of multiple analytes comprising:
(a) a chip with a flat surface, said flat surface having a plurality of reaction areas defined thereon, each said reaction area further containing at least one array of discrete test sites, each said test site having a test molecule immobilized thereon, and
(b) a divider sheet having a plurality of holes defined within a frame, each said hole corresponding to a reaction area, said frame adapted for reversible coupling onto said flat surface such that a plurality of leak-proof wells are created therebetween with each reaction area forming the base of a corresponding well and the frame of said sheet forming dividing walls between neighbouring wells.

6. A device according to claim 5 where in said test molecule is an antigen or an antibody.

7. A method of analysing multiple analytes in a sample comprising:
(a) defining a plurality of discrete and spatially separate test sites on at least one test area on a test surface;
(b) immobilizing a test molecule onto each of said test sites such that a plurality of test molecules are immobilized thereon;
(c) providing a fluid-proof barrier around each said test area;
(d) contacting each said test area with the appropriate test reagents and the sample for analysis;
(e) removing unreacted sample and reagent;
(f) removing said barrier; and
(g) analysing said test surface for reaction between the analyte in said sample and said test molecule.

8. A method of analysing multiple analytes from the same sample according to claim 7 wherein said test surface is one side of a chip, said test molecule is an antibody or a ligand, and said steps (d) and (e) further comprises the steps of:
incubating said sample with the immobilized test molecules in each test area for said analytes to bind thereto, said analyte being a cross-reacting ligand or antibody;
removing unbound material;
adding a mixture of appropriate secondary molecules to said reaction area and incubating therein, each said secondary molecule having a signal molecule coupled thereto, said secondary molecule further adapted to bind specifically with the appropriate analyte;
removing unbound secondary molecules; and
detecting the presence of said signal molecule on each of said test sites.

9. A method according to claim 7 wherein said test molecule is a hormones, drug, receptor, enzyme, nucleic acid, antibiotic or ligand.

10. A method according to claim 7 wherein said secondary molecule is a secondary antibody that cross-reacts with said appropriate analyte; said signal molecule is a fluorescence molecule capable of emitting an emission photon upon excitation of an excitation photon, and said step of detecting the presence of said signal molecule further comprises the steps of exciting said signal molecule with said excitation light, and using a detector to detect emission of the emission photon using a fluorescence detector.

11. A method according to claim 8 wherein said signal molecule is an enzyme, and said step of detecting the presence of said signal molecule further comprises the steps of providing an appropriate substrate for said enzyme and incubating therewith, said enzyme adapted to convert said substrate into a coloured product that is deposited onto said test site.

12. A method according to claim 8 wherein said secondary molecule is a secondary antibody that cross reacts with said appropriate analytes, said signal molecule is biotin, and said step of detecting the present of said signal molecule further comprises contacting said signal molecule with strepavidin or avidin to allow binding thereto; removing unbound strepavidin or avidin; contacting said bound strepavidin or avidin with a solution containing secondary biotin, said secondary biotin conjugated with a fluorescence molecule, said fluorescence molecule capable of emitting an emission photon; shining an excitation photon onto said chip and using a detector to detect the emission of an emission photon from said fluorescence molecule.

13. A method according to claim 6 wherein said test molecule is selected from a group comprising Hepatitis A viral Antigen, Hepatitis C viral Antigen, Hepatitis D viral antigen, Hepatitis B surface antigen, Hepatitis B ‘e’ antigen, Hepatitis B Core antigen and Hepatitis E viral Antigen, antibody against Hepatitis B surface antigen, antibody against Hepatitis B ‘e’ antigen, antibody against alpha-fetoprotein antigen and antibody against Hepatitis D viral antigen.

14. A Method according to claim 6 wherein said test molecule is selected from a group comprising Toxoplasma gondii, Rubella virus, Cytomegalovirus, Herpes Simplex Virus I and Herpes Simplex Virus II.

15. A method according to claim 6 where in said test molecule is selected from a group comprising: anti-alpha-fetoprotein, anti-gamma-glutamyl transferase isozyme I antibody, anti-gamma-glutamyl transferase isozyme I′ antibody, anti-gamma-glutamyl transferase isozyme II antibody, anti-gamma-glutamyl transferase isozyme II′ antibody, anti-des-gamma carboxy prothrombin antibody, anti-alpha-L-fucosidase antibody, anti-5′ nucleotide phosphodiesterases isozyme V antibody, anti-glutathione S-transferase-placental form antibody, anti-apha-1-antitrypsin antibody, anti-ferritin antibody, anti-acidic isoferritin antibody, and anti-hepatitis B surface antigen antibody.

16. A method according to claim 6 where in said test molecule is selected from a group consisting of single stranded nucleic acid, double stranded nucleic acid, the SMITH antigen; acetylcholine receptor, thyroglobulin, microsomal antigen, abnormal human IgG and red blood cell protein.
